(19) United States
(12) Patent Application Publication
(10) Pub. No.: US 2005/0043525 A1
(43) Pub. Date: Feb. 24, 2005

(54) GlutRNAGln amidotransferase - a novel essential translational component

(76) Inventor: Dieter Soll, Hamden, CT (US)

Correspondence Address: DARBY & DARBY P.C., P. O. BOX 5257, NEW YORK, NY, 10150-5257, US

(21) Appl. No.: 10/661,399

(22) Filed: Sep. 12, 2003

Related U.S. Application Data
(63) Continuation of application No. 09/355,622, filed on Sep. 23, 1999, now abandoned.
(60) Provisional application No. 60/037,275, filed on Feb. 3, 1997.

(51) Int. Cl. C07H 21/04

(52) U.S. Cl. 536/23.2

(57) ABSTRACT

The present method provides the amino acid sequence and encoding nucleic acid sequence of GlutRNAGln amidotransferase (AdT), a protein that is essential for protein translation. The AdT proteins and encoding nucleic acid molecules herein described can be used as targets for identifying agents that block translations. Such agents can be used as an antimicrobial, antifungal or herbicide agent.

### CROSS REFERENCE TO RELATED APPLICATIONS

This application is related to U.S. Provisional Application Ser. No. 60/037,275; filed Feb. 3, 1997, which is herein incorporated by reference in its entirety.

### FIELD OF THE INVENTION

The present invention is in the field of inhibitors of protein tabulation, particularly translation of proteins within microorganisms and organelles. This invention relates to newly identified polynucleotides and polypeptides, and their production and uses, as well as their variants, agonists and antagonists, and their uses.

In particular, in these and in other regards, the invention relates to novel polynucleotides and polypeptides of the Glu-tRNA^{Gln} Amidotransferase family, hereinafter referred to as “Glu-tRNA^{Gln} AdT” or “AdT”. The present invention further provides methods and compositions for use in identifying and using protein translation inhibitors as antibacterial, antifungal or herbicidal agents.

### BACKGROUND OF THE INVENTION

Prior to their incorporation into protein, amino acids are chemically linked to small RNA molecules called transfer RNA (tRNA). For each of the 20 different amino acids, a specific enzyme catalyzes its linkage to the 3′ end of its specific tRNA molecule. While the general mechanism of protein biosynthesis (the translation process) is conserved throughout the living kingdom there exist two different pathways for the formation of GlntRNA^{Gln}. While the two pathways for GlntRNA^{Gln} formation are evolutionarily conserved, the reason for existence of the different pathways is as yet not known. In gram-negative eubacteria and in the cytoplasm of eukaryotic cells the enzyme glutaminyl-tRNA synthetase (GlnRS) acylates glutamine directly to the cognate tRNA to provide GlntRNA^{Gln}. Interestingly, GlnRS is not detectable in several biological systems. In certain organisms and organelles including the archae, gram-positive eubacteria, mitochondria and chloroplasts a different pathway of GlntRNA^{Gln} formation, a transamidation pathway is operative (Curnow et al. (1996) Nature 382: 589-590; Curnow et al. (1997) Proc. Natl. Acad. Sci. USA 94(22):11819-11826; Schön et al. (1988) Biochimie 70(3):391-394; Wilcox & Nirenberg (1968) Proc. Natl. Acad. Sci. USA 61(1):229-236; Schön et al. (1988) Nature 331:187-190. This pathway (depicted in FIG. 1) is initiated by misacylation of tRNA^{Gln} by glutamyl-tRNA synthetase (GluRS) forming GlutRNA⁰. The incorrectly charged tRNA is then converted to GlntRNA^{Gln} by GlutRNA^{Gln} amidotransferase (AdT). AdT catalyzes the amidation of glutamate to glutamine only when the glutamate is covalently attached to tRNAGln. It has been shown that the partially purified GlutRNA^{Gln} amidotransferase activity from Bacillus megaterium in the presence of ATP, Mg++, and an amide-nitrogen donor (glutamine) will carry out the amidation of GlutRNA^{Gln} to GlntRNA^{Gln} (Wilcox & Nirenberg, 1968). Subsequent work demonstrated, in vitro, that the amidation proceeds through the activated intermediate (phospho-GlutRNA^{Gln}) (Wilcox (1969) Cold Spring Harb. Symp. Quant. Biol. 34:521-528, Wilcox (1969) Eur J. Biochem 11(3):405-412). Since the initial aminoacylation product, GlutRNA^{Gln}, would be toxic to the cell due to the fact that it would result in faulty protein translation, it must be converted to the correctly charged tRNA.

It appears that this pathway is the primary source of GlntRNA^{Gln} within these cells and may act as a regulatory mechanism for glutamine metabolism. Evolutionarily, it has been suggested that glutamine was the last amino acid formed. Therefore it may be postulated that cells which employ the transamidation pathway utilized the gene encoding GluRS to generate the AdT. Likewise, in the cells in which the direct glutaminylation pathway operates, the enzyme GlnRS may have evolved from a GluRS gene duplication (Rogers & Soil (1995) J. Mol. Evol. 40 (5) p476-81). This is reasonable since both enzymes are required to specifically recognize and bind tRNAGln and free glutamine. However, database searches and, in particular, a detailed analysis of the Mycoplasma genome (Fraser et a). (1995) Science 270(5235):397-403), the only gram-positive organism sequenced and published to date, have shown no significant homologies to GluRS and GlnRS in the currently available sequence information. Thus, the amidotransferase may not have significant homology to the aminoacyl-tRNA synthetases. Despite the unquestioned evolutionary and biochemical significance in understanding this system, there have been very few investigations of this enzyme to date (Wilcox & Nirenberg, 1968; Wilcox, 1969; Strauch et al. (1988) J. Bacterol. 170:916-920; and Jahn (1990) J. Biol. Chem. 265(14):8059-64).

### SUMMARY OF THE INVENTION

The present invention is based, in part, on the isolation and characterization of a heterotrimeric protein designated AdT that is involved in generating GlntRNA^{Gln} from GlutRNA^{Gln}. This invention further provides polypeptides that have been identified as novel AdT polypeptides by homology between the amino acid sequence of GlutRNA^{Gln} AdT and a known amino acid sequence.

This invention farther provides polynucleotides that encode AdT polypeptides. In particular, this invention provides the polynucleotide sequence encoding GlutRNA^{Gln} AdT comprising the sequence set out in FIG. 3 (SEQ ID NO:1), or a variant thereof, such as naturally occurring allelic variants of AdT and polypeptides encoded thereby. Thus, this invention provides polynucleotides that hybridize to AdT polynucleotide sequences, particularly under stringent conditions.

This invention provides GlutRNA^{Gln} AdT protein from B. subtilis comprising the amino acid sequences encoded by the nucleotide sequence of FIG. 3 (SEQ ID NOS:1, 3, 5 and 7), as well as biologically, diagnostically, prophylactically, clinically or therapeutically useful variants thereof, and compositions comprising the same. Particularly preferred variants include AdT polypeptides encoded by naturally occurring alleles of the AdT gene. Methods for producing the aforementioned AdT polypeptides are also provided by this invention.

The invention also provides isolated nucleic acid molecules encoding AdT, particularly B. subtilis AdT, including mRNAs, cDNAs, and genomic DNAs, including biologically, diagnostically, prophylactically, clinically or therapeutically useful variants thereof, and compositions comprising the same.

In accordance with yet another aspect of the invention, there are provided inhibitors to such AdT polypeptides, useful as antibacterial agents, antifungal agents and herbicides. Thus, the present invention provides compositions and methods for use in identifying agonists and antagonists of the AdT protein.

This invention provides compositions and methods for (i) assessing AdT expression, (ii) treating disease, for example, diseases associated with excessive or deficient amounts of available AdT, (iii) assaying genetic variation, and (iv) and administering an AdT polypeptide or polynucleotide to a cell or to a multicellular organism to raise an immunological response. In certain preferred embodiments of this aspect of the invention there are provided antibodies against AdT polypeptides.

This invention also provides compositions and methods for protecting plants, especially crop plants. For example, this invention provides antagonists of AdT which are useful as herbicides, as well as the herbicidal compositions which include such inhibitors of AdT. This invention also provides non-inhibited mutants of AdT and functional derivatives thereof which are resistant to inhibition from certain herbicides, especially herbicides containing inhibitors of AdT. The polynucleotides coding for the non-inhibited AdT can be placed in plants by various transformation methods so as to render the plants tolerant or resistant to certain herbicides containing inhibitors of AdT. Therefore, methods of treating weeds utilizing the application of AdT inhibitors to transgenic plants containing the non-inhibited mutants of AdT are also encompassed by this invention.

Various changes and modifications within the spirit and scope of the disclosed invention will become readily apparent to those skilled in the art from reading the following description and from reading the other parts of the present disclosure.

### BRIEF DESCRIPTION OF THE DRAWINGS

FIG. 1 shows the transamidation pathway for the formation of GlntRNA^{Gln}.

FIG. 2 shows the gene arrangement of the AdT gene.

FIG. 3 shows the nucleic acid sequence of the AdT protein from B. subtilis.

### DESCRIPTION OF THE INVENTION

I. General Description

The present invention is based, in part, on the identification and characterization of a heterotrimeric protein that is responsible for generating GlntRNA within a cell, herein after the AdT protein. The present invention specifically provides the amino acid sequences of each of the three subunits of an AdT protein isolated from B. subtilis, as well as nucleotide sequences that encode the AdT protein. The AdT protein and nucleic acid molecules can serve as targets in methods for identifying agents for use in inhibiting protein synthesis, particularly antimicrobial, antifungal and herbicide agents.

II. Specific Embodiments

A. AdT Protein

Prior to the present invention the art had taught that there was an enzyme involved in converting GlutRNA^{Gln} to GlntRNA^{Gln}. However the isolation and characterization of the protein responsible for generating GlntRNA remained unknown. The present invention provides, in part, the amino acid sequences of the three subunits of the B. subtilis AdT protein. Quite unexpectedly, this AdT protein was found to be a heterotrimeric protein.

In one embodiment, the present invention provides the ability to produce a previously unknown protein using the cloned nucleic acid molecules herein described or by synthesizing a protein having the amino acid sequence herein disclosed.

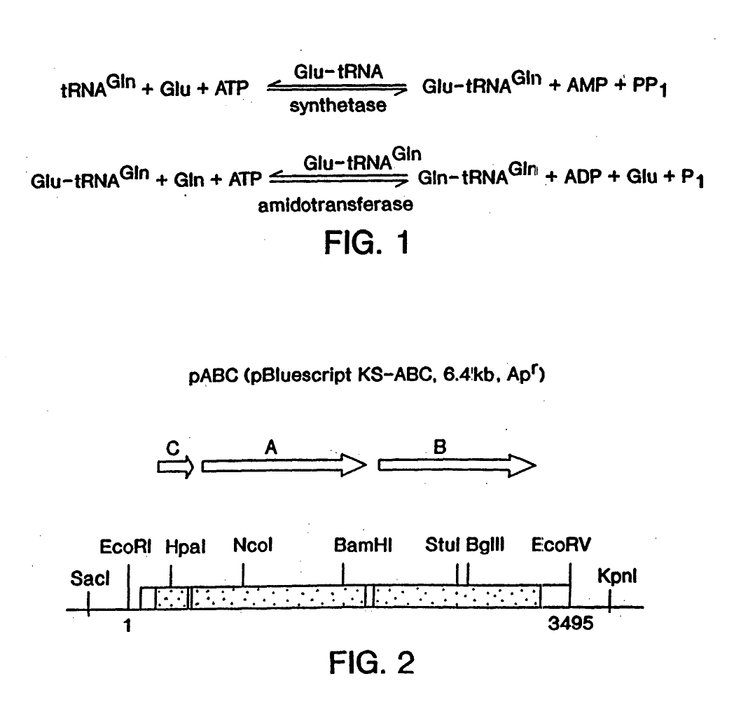

As used herein, the AdT protein refers to a protein that has the amino acid sequence B. subtilis AdT encoded by the polynucleotide of FIG. 1, allelic variants thereof and conservative substitutions thereof that have AdT activity. The AdT protein is comprised of 3 subunits: the A (SEQ ID NO:4), B (SEQ ID NO:6) and C (SEQ ID NO:8) subunits, referred to herein collectively as aAdT, bAdT and cAdT subunits, respectively. For the sake of convenience, the collective subunits will be referred to as the AdT protein or the AdT protein of the present invention. A skilled artisan can readily recognize within the context whether a single subunit or the collective protein is being referred to.

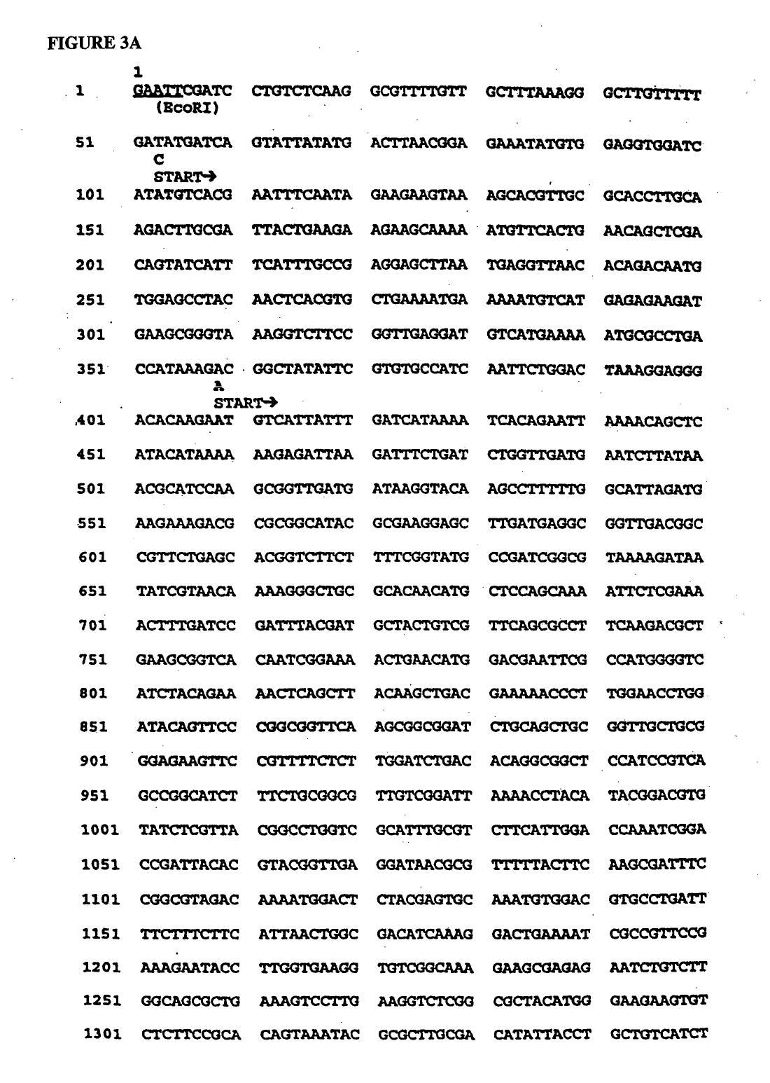

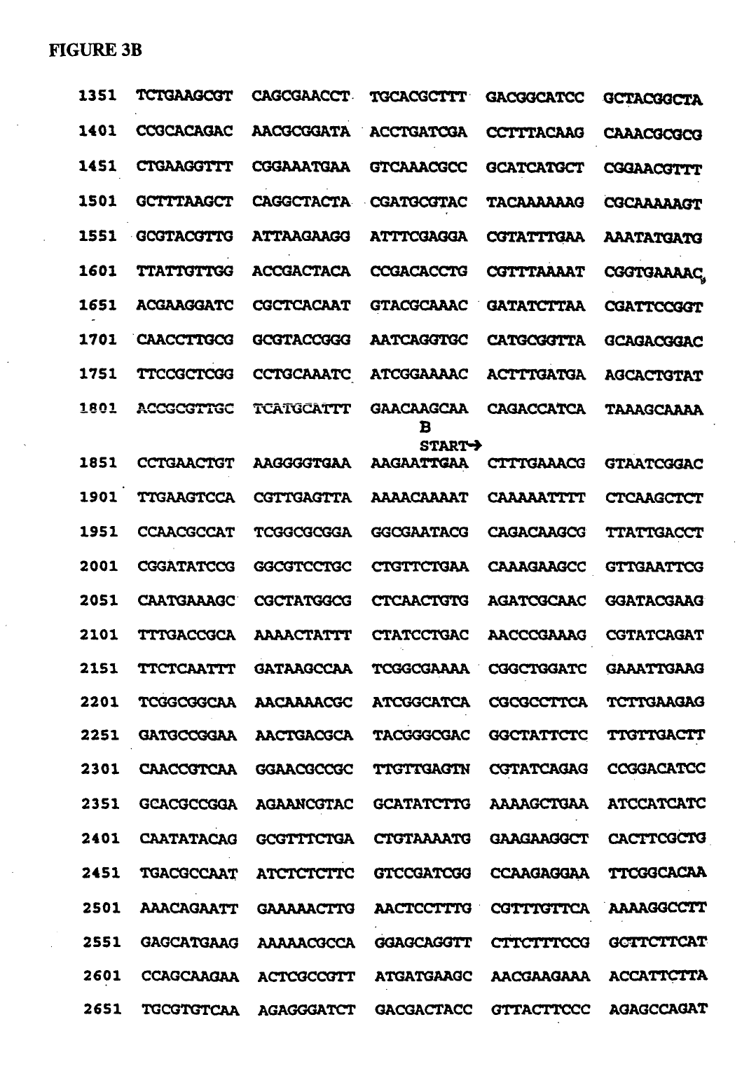

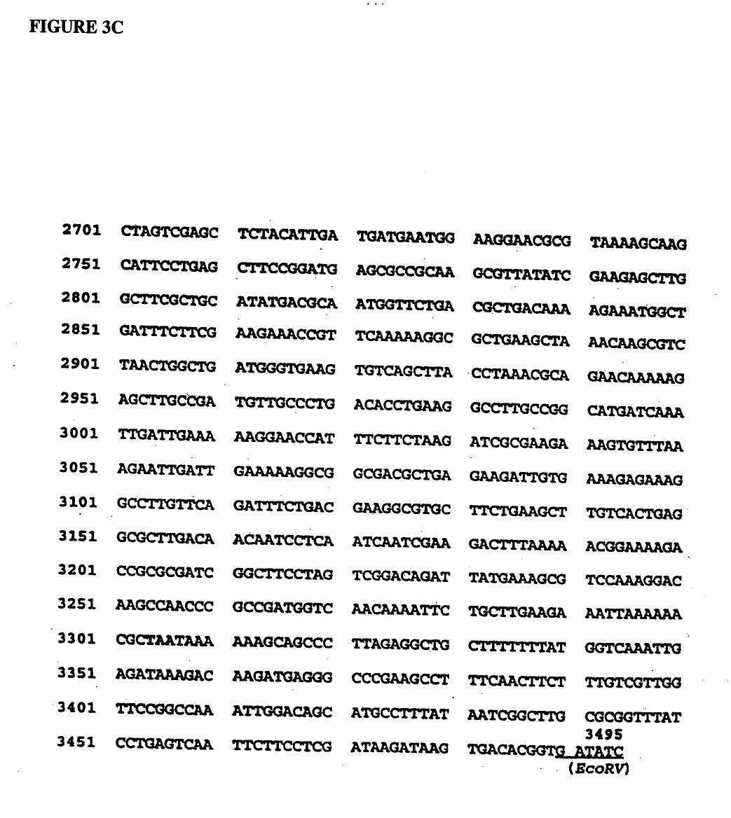

The polypeptides of the invention include the polypeptides encoded by SEQ ID NO:1 (FIG. 3) as well as polypeptides and fragments, particularly those which have the biological activity of AdT and also those which have at least 70% sequence identity to the polypeptides encoded by SEQ ID NO:1 or the relevant portion, preferably at least 80% identity to the polypeptides encoded by SEQ ID NO:1, and more preferably at least 90% similarity (more preferably at least 90% identity) to the polypeptides encoded by SEQ ID NO:1 and still more preferably at least 95% similarity (still more preferably at least 95% identity) to the polypeptides encoded by SEQ ID NO:1 and also include portions of such polypeptides.

The AdT proteins of the present invention include the specifically identified and characterized variant herein described as well as allelic variants, conservative substitution variants and homologues that can be isolated/generated and characterized without undue experimentation following the methods outlined below. For the sake of convenience, all AdT proteins will be collectively referred to as the AdT proteins or the AdT proteins of the present invention. The term “AdT proteins” includes all naturally occurring allelic variants of the B. subtilis AdT protein that possess normal AdT activity. In general, allelic variants of the AdT protein will have a slightly different amino acid sequence than that specifically encoded by SEQ ID NO:1 but will be able to convert GlutRNA to GlntRNA. Allelic variants, though possessing a slightly different amino acid sequence than those recited above, will posses the ability to generate GlntRNA Typically, allelic variants of the AdT protein will contain conservative amino acid substitutions from the AdT sequences herein described or will contain a substitution of an amino acid from a corresponding position in an AdT homologue (an AdT protein isolated from an organism other than B. subtilis).

The AdT proteins of the present invention are preferably in isolated form. As used herein, a protein is said to be isolated when physical, mechanical or chemical methods are employed to remove the AdT protein from cellular constituents that are normally associated with the protein. A skilled artisan can readily employ standard purification methods to obtain an isolated AdT protein. One purification scheme is outlined in Example 1. The nature and degree of isolation will depend on the intended use.

The cloning of an AdT encoding nucleic acid molecule makes it possible to generate defined fragments of the AdT proteins of the present invention. As discussed below, fragments of the AdT proteins of the present invention are particularly useful in generating subunit specific antibodies, in identifying agents that bind to a AdT protein and in isolating homologues of the B. subtilis AdT protein.

Fragments of the AdT proteins can be generated using standard peptide synthesis technology and the amino acid sequences disclosed herein. Alternatively, recombinant methods can be used to generate nucleic acid molecules that encode a fragment of the AdT protein.

Fragments of the AdT protein subunits that contain particularly interesting structures can be identified using art-known methods such as immunogenicity, Chou-Fasman, Garnier-Robson, Kyte-Doolittle, Eisenberg, Karplus-Schultz or Jameson-Wolf analysis. Fragments containing such residues are particularly useful in generating subunit specific anti-AdT antibodies.

As described below, members of the AdT family of proteins can be used for, but are not limited to: 1) a target to identify agents that block or stimulate AdT activity, 2) a target or bait to identify and isolate binding partners that bind an AdT protein, 3) identifying agents that block or stimulate the activity of an AdT protein and 4) an assay target to identify AdT mediated activity or disease.

B. Anti-AdT Antibodies

The present invention further provides antibodies that selectively bind one or more of the AdT proteins of the present invention, or to a specific subunit of an AdT protein of the present invention. The most preferred antibodies will bind to either an entire heterotrimeric AdT protein but not to an isolated subunit or will bind to an isolated subunit but not to the assembled trimeric protein. Anti-AdT antibodies that are particularly contemplated include monoclonal and polyclonal antibodies as well as fragments containing the antigen binding domain and/or one or more complement determining regions of these antibodies.

Antibodies are generally prepared by immunizing a suitable mammalian host using an AdT protein, or fragment, in isolated or immunoconjugated form (Harlow, Antibodies, Cold Spring Harbor Press, NY (1989)). Regions of the AdT protein that show immunogenic structure can readily be identified using art-known methods. Other important regions and domains can readily be identified using protein analytical and comparative methods known in the art.

Fragments containing these residues are particularly suited in generating specific classes of anti-AdT antibodies.

Methods for preparing a protein for use as an immunogen and for preparing immunogenic conjugates of a protein with a carrier such as BSA, KLH, or other carrier proteins are well known in the art. In some circumstances, direct conjugation using, for example, carbodiimide reagents may be used; in other instances linking reagents such as those supplied by Pierce Chemical Co., Rockford, Ill., may be effective.

Administration of an AdT immunogen is conducted generally by injection over a suitable time period and with use of a suitable adjuvant, as is generally understood in the art. During the immunization schedule, titers of antibodies can be taken to determine adequacy of antibody formation.

While the polyclonal antisera produced in this way may be satisfactory for some applications, for pharmaceutical compositions, monoclonal antibody preparations are preferred. Immortalized cell lines which secrete a desired monoclonal antibody may be prepared using the standard method of Kohler and Milstein or modifications which effect immortalization of lymphocytes or spleen cells, as is generally known. The immortalized cell lines secreting the desired antibodies are screened by immunoassay in which the antigen is the AdT protein or peptide fragment. When the appropriate immortalized cell culture secreting the desired antibody is identified the cells can be cultured either in vitro or by production in ascites fluid.

The desired monoclonal antibodies are then recovered from the culture supernatant or from the ascites supernatant. Fragments of the monoclonals or the polyclonal antisera which contain the immunologically significant portion can be used as antagonists, a well as the intact antibodies. Use of immunologically reactive fragments, such as the Fab, Fab′, of F(ab′)₂ fragments is often preferable, especially in a therapeutic context, as these fragments are generally less immunogenic than the whole immunoglobulin.

The antibodies or fragments may also be produced, using current technology, by recombinant means. Regions that bind specifically to the desired regions of the transporter can also be produced in the context of chimeric or CD, grafted antibodies of multiple species origin.

As described below, anti-AdT antibodies are useful as modulators of AdT activity, are useful in immunoassays for detecting AdT expression/activity and for purifying homologies of the B. subtilis AdT protein.

C. AdT Encoding Nucleic Acid Molecules

As described above, the present invention is based, in part, on isolating nucleic acid molecules from B. subtilis that encode the three subunits of the AdT protein. Accordingly, the present invention further provides nucleic acid molecules that encode the AdT protein, as herein defined, preferably in isolated form. For convenience, all AdT encoding nucleic acid molecules will be referred to as AdT encoding nucleic acid molecules, the AdT genes, or AdT. The nucleotide sequence of the B. subtilis nucleic acid molecule that encodes each of the subunits of AdT is provided in SEQ ID NO: 1. The start and stop codons for each of subunits A (SEQ ID NO:4), B (SEQ ID NO:6) and C (SEQ ID NO:8) are designated in the nucleotide sequence for AdT provided in FIG. 3.

Further preferred embodiments of the invention are polynucleotides that are at least 70% sequence identical over their entire length to a polynucleotide encoding AdT polypeptides having an amino acid sequence encoded by SEQ ID NO:1, and polynucleotides which are complementary to such polynucleotides. Alternatively, most highly preferred are polynucleotides that comprise a region that is at least 80% identical over their entire length to a polynucleotide encoding AdT polypeptide and polynucleotides complementary thereto. In this regard, polynucleotides at least 90% identical over their entire length to the same are particularly preferred, and among these particularly preferred polynucleotides, those with at least 95% are especially preferred. Furthermore, those with at least 97% are highly preferred among those with at least 95%, and among these those with at least 98% and at least 99% are particularly highly preferred, with at least 99% being the more preferred.

The invention further relates to variants of the herein above described polynucleotides which encode for variants of the polypeptides having the deduced amino acid sequences of SEQ ID NO:1.

Variants that are fragments of the polypeptides of the invention may be employed for producing the corresponding full-length polypeptide by peptide synthesis; therefore, these variants may be employed as intermediates for producing the full-length polypeptides. Variants that are fragments of the polynucleotides of the invention may be used to synthesize full-length polynucleotides of the invention. Variants that are fragments of the polynucleotides of the invention may be used to synthesize full-length polynucleotides of the invention. Such methods are widely available, such as those disclosed in WO 97/26340 and WO 97/38716.

A fragment is a variant polypeptide having an amino acid sequence that entirely is the same as part but not all of the amino acid sequence of the aforementioned polypeptides, AdT polypeptides fragments may be “free-standing,” or comprised within a larger polypeptide of which they form a part or region, most preferably as a single continuous region, a single larger polypeptide.

Further particularly preferred embodiments are polynucleotides encoding AdT variants, which have the amino acid sequence of the AdT polypeptides encoded by SEQ ID NO:1 in which several, a few, 10 to 15, 5 to 10, 1 to 5, 1 to 3, 2, 1 or no amino acid residues are substituted, deleted or added, in any combination. Especially preferred among these are silent substitutions, additions and deletions, which do not alter the properties and activities of AdT.

As used herein, a “nucleic acid molecule” is defined as an RNA or DNA molecule that encodes a peptide as defined above, or is complementary to a nucleic acid sequence encoding such peptides. Particularly preferred nucleic acid molecules will have a nucleotide sequence identical to or complementary to the B. subtilis DNA sequences herein disclosed. Specifically contemplated are genomic DNA, polycistronic mRNA and antisense molecules, as well as nucleic acids based on an alternative backbone or including alternative bases, whether derived from natural sources or synthesized. Such nucleic acid molecules, however, are defined further as being novel and unobvious over any prior art nucleic acid molecules encoding non-AdT proteins isolated from organisms other than B. subtilis.

As used herein, a nucleic acid molecule is said to be “isolated” when the nucleic acid molecule is substantially separated from contaminant nucleic acid molecules that encode polypeptides other than AdT. A skilled artisan can readily employ nucleic acid isolation procedures to obtain an isolated AdT encoding nucleic acid molecule.

The present invention further provides fragments of the AdT encoding nucleic acid molecules of the present invention. As used herein, a fragment of an AdT encoding nucleic acid molecule refers to a small portion of the entire protein encoding sequence. The size of the fragment will be determined by its intended use. For example, if the fragment is chosen so as to encode an active portion of the AdT protein, such an active domain or effector binding site, then the fragment will need to be large enough to encode the functional region(s) of the AdT protein. If the fragment is to be used as a nucleic acid probe or PCR primer, then the fragment length is chosen so as to obtain a relatively small number of false positives during probing/priming. Fragments of the B. subtilis AdT nucleic acid molecule that are particularly useful as selective hybridization probes or PCR can be readily determined using art-known methods.

Fragments of the AdT encoding nucleic acid molecules of the present invention (i.e., synthetic oligonucleotides) that are used as probes or specific primers for the polymerase chain reaction (PCR), or to synthesize gene sequences encoding AdT proteins, can easily be synthesized by chemical techniques, for example, the phosphotriester method of Matteucci, et al., J Am Chem Soc (1981) 103:3185-3191 or using automated synthesis methods. In addition, larger DNA segments can readily be prepared by well known methods, such as synthesis of a group of oligonucleotides that define various modular segments of the AdT gene, followed by ligation of oligonucleotides to build the complete modified AdT gene.

The AdT encoding nucleic acid molecules of the present invention may further be modified so as to contain a detectable label for diagnostic and probe purposes. As described above, such probes can be used to identify nucleic acid molecules encoding other allelic variants or homologues of the AdT proteins and as described below, such probes can be used to diagnose the presence of a AdT protein as a means for diagnosing a pathological condition caused by AdT mediated translation. A variety of such labels are known in the art and can readily be employed with the AdT encoding molecules herein described. Suitable labels include, but are not limited to, biotin, radiolabeled nucleotides, biotin, and the like. A skilled artisan can employ any of the art-known labels to obtain a labeled AdT encoding nucleic acid molecule.

D. Isolation of Other AdT Encoding Nucleic Acid Molecules

The identification of the AdT protein from B. subtilis and the corresponding nucleic acid molecules, has made possible the identification of and isolation of AdT proteins from organisms other than B. subtilis, hereinafter referred to collectively as AdT homologues. The preferred source of the AdT homologues are pathogenic microorganisms such as bacteria and fungi, as well as plants in which it is desirable to control growth. The most preferred sources are gram positive bacteria, pathogenic fungi and plant organelles such as chloroplasts.

Essentially, a skilled artisan can readily use the amino acid sequence of the B. subtilis AdT protein to generate antibody probes to screen expression libraries prepared from cells. Typically, polyclonal antiserum from mammals such as rabbits immunized with the purified protein (as described below) or monoclonal antibodies can be used to probe an expression library, prepared from a target organism, to obtain the appropriate coding sequence for AdT protein homologue. The cloned cDNA sequence can be expressed as a fission protein, expressed directly using its own control sequences, or expressed by constructing an expression cassette using control sequences appropriate to the particular host used for expression of the enzyme.

Alternatively, a portion of the AdT encoding sequence herein described can be synthesized and used as a probe to retrieve DNA encoding a member of the AdT family of proteins from organisms other than B. subtilis. Oligomers containing approximately 18-20 nucleotides (encoding about a 6-7 amino acid stretch) are prepared and used to screen genomic DNA or cDNA libraries to obtain hybridization under stringent conditions or conditions of sufficient stringency to eliminate an undue level of false positives. This method can be used to identify and isolate altered and variants of the AdT encoding sequences.

Additionally, pairs of oligonucleotide primers can be prepared for use in a polymerase chain reaction (PCR) to selectively amplify/clone an AdT-encoding nucleic acid molecule, or fragment thereof. A PCR denature/anneal/extend cycle for using such PCR primers is well known in the art and can readily be adapted for use in isolating other AdT encoding nucleic acid molecules. Regions of the B. subtilis AdT gene that are particularly well suited for use as a probe or as primers can be readily identified. In general, the preferred primers will flank one or more of the subunit encoding regions of the B. subtilis AdT gene.

Homologues of the herein disclosed AdT proteins will share homology. In general, nucleic acid molecules that encode AdT homologues will hybridize to the B. subtilis sequences under high stringency. Such sequences will typically contain at least 70% homology, preferably at least 80%, most preferably at least 90% homology to the B. subtilis sequences.

E. Recombinant DNA Molecules Containing Arm AdT Encoding Nucleic Acid Molecule

The present invention further provides recombinant DNA molecules that contain one or more of the AdT encoding sequences herein described, or a fragment of the herein-described nucleic acid molecules. As used herein, an recombinant DNA molecule is a DNA molecule that has been subjected to molecular manipulation in vitro. Methods for generating recombinant DNA molecules are well known in the art, for example, see Sambrook et al., Molecular Cloning (1989). In the preferred recombinant DNA molecules, an AdT encoding DNA sequence that encodes an AdT protein, or AdT subunit is operably linked to one or more expression control sequences and/or vector sequences. The recombinant DNA molecule can encode either a single subunit of the AdT protein, or can encode an operon that contains all three of the AdT subunits.

The choice of vector and/or expression control sequences to which one of the AdT encoding sequences of the present invention is operably linked depends directly, as is well known in the art, on the functional properties desired e.g., protein expression, and the host cell to be transformed. A vector contemplated by the present invention is at least capable of directing the replication or insertion into the host chromosome, and preferably also expression, of an AdT encoding sequence included in the recombinant DNA molecule.

Expression control elements that are used for regulating the expression of an operably linked protein encoding sequence are known in the art and include, but are not limited to, inducible promoters, constitutive promoters, secretion signals, enhancers, transcription terminators and other regulatory elements. Preferably, an inducible promoter that is readily controlled, such as being responsive to a nutrient in the host cell's medium, is used.

In one embodiment, the vector containing an AdT encoding nucleic acid molecule will include a prokaryotic replicon, i.e., a DNA sequence having the ability to direct autonomous replication and maintenance of the recombinant DNA molecule intrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or viral promoter capable of directing the expression (transcription and translation) of the AdT encoding sequence in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 available from Biorad Laboratories (Richmond, Calif.), pPL and pKK223 available from Pharmacia, Piscataway, N.J.

Expression vectors compatible with eukaryotic cells, preferably those compatible with vertebrate cells, can also be used to variant recombinant DNA molecules that contain an AdT encoding sequence. Eukaryotic cell expression vectors are well known in the art and are available from several commercial sources. Typically, such vectors are provided containing convenient restriction sites for insertion of the desired DNA segment. Typical of such vectors are PSVL and pKSV-10 (Pharmacia), pBPV-1/pML2d (International Biotechnologies, Inc.), pTDT1 (ATCC, #31255), the vector pCDM8 described herein, and the like eukaryotic expression vectors.

Eukaryotic cell expression vectors used to construct the recombinant DNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. Southern et al., J Mol Anal Genet (1982) 1:327-341. Alternatively, the selectable marker can be present on a separate plasmid, and the two vectors are introduced by cotransfection of the host cell, and selected by culturing in the presence of the appropriate drug for the selectable marker.

F. Host Cells Containing an Exogenously Supplied AdT Encoding Nucleic Acid Molecule

The present invention further provides host cells transformed with a nucleic acid molecule that encodes an AdT protein of the present invention, either the entire heterotrimeric protein or one or more subunits. The host cell can be either prokaryotic or eukaryotic. Eukaryotic cells useful for expression of an AdT protein are not limited, so long as the cell line is compatible with cell culture methods and compatible with the propagation of the expression vector and expression of an AdT gene. Preferred eukaryotic host cells include, but are not limited to, yeast, insect and mammalian cells, preferably vertebrate cells such as those from a mouse, rat, monkey or human fibroblastic cell line, the most preferred being cells that do not naturally express an AdT protein.

Any prokaryotic host can be used to express an AdT-encoding recombinant DNA molecule. The preferred prokaryotic host is E. coli.

Transformation of appropriate cell hosts with an recombinant DNA molecule of the present invention is accomplished by well known methods that typically depend on the type of vector used and host system employed. With regard to transformation of prokaryotic host cells, electroporation and salt treatment methods are typically employed, see, for example, Cohen et al., Proc Acad Sci USA (1972) 69:2110; and Maniatis et al., Molecular Cloning, A Laboratory Manual, Cold Spring Harbor Laboratory, Cold Spring Harbor, N.Y. (1982). With regard to transformation of vertebrate cells with vectors containing recombinant DNAs, electroporation, cationic lipid or salt treatment methods are typically employed, see, for example, Graham et al., Virol (1973) 52:456; Wigler et al., Proc Natl Acad Sci USA (1979) 76:1373-76.

Successfully transformed cells, i.e., cells that contain an recombinant DNA molecule of the present invention, can be identified by well known techniques. For example, cells resulting from the introduction of an recombinant DNA of the present invention can be cloned to produce single colonies. Cells from those colonies can be harvested, lysed and their DNA content examined for the presence of the recombinant DNA using a method such as that described by Southern, J Mol Biol (1975) 98:503, or Berent et al., Biotech (1985) 3:208 or the proteins produced from the cell assayed via an immunological method.

G. Production of an AdT Protein Using an Recombinant DNA Molecule

The present invention further provides methods for producing an AdT protein that uses one of the AdT encoding nucleic acid molecules herein described. In general terms, the production of a recombinant AdT protein typically involves the following steps.

First, a nucleic acid molecule is obtained that encodes an AdT protein, such as the nucleic acid molecule depicted in FIG. 3 (SEQ ID NO:1) or an AdT subunit. The AdT encoding nucleic acid molecule is then preferably placed in an operable linkage with suitable control sequences, as described above, to generate an expression unit containing the AdT encoding sequence. The expression unit is used to transform a suitable host and the transformed host is cultured under conditions that allow the production of the AdT protein. Optionally the AdT protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in an appropriate host. The construction of expression vectors that are operable in a variety of hosts is accomplished using an appropriate combination of replicons and control sequences. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with AdT encoding sequences to produce an AdT protein.

H. Identification of Agents that Bind to an AdT Protein

Another embodiment of the present invention provides methods for identifying agents that are agonists or antagonists of the AdT proteins herein described.

Specifically, agonists and antagonists of an AdT protein can be identified by the ability of the agent to bind to an AdT protein and/or the ability to inhibit AdT activity. Activity assays for AdT activity and binding assays using an AdT protein are suitable for use in high through-put screening methods.

In detail, in one embodiment, an AdT protein is mixed with an agent. After mixing under conditions that allow association of AdT protein with the agent, the mixture is analyzed to determine if the agent bound the AdT protein. Agonists and antagonists are identified as being able to bind to an AdT protein. Alternatively or consecutively, as described below, AdT activity can be directly assessed as a means for identifying agonists and antagonists of AdT activity.

The AdT protein used in the above assay can be: an isolated and filly characterized protein, a single subunit of an AdT protein, a partially purified protein, a cell that has been altered to express an AdT protein or a fraction of a cell that has been altered to express an AdT protein. Further, the AdT protein can be the entire AdT protein, a specific fragment of the AdT protein or a single subunit of the AdT protein. It will be apparent to one of ordinary skill in the art that so long as the AdT protein can be assayed for agent binding, e.g., by a shift in molecular weight or activity, as described in the Examples, the present assay can be used. The AdT protein is particularly well suited for high through-put screening methods.

The source of the AdT protein will be based on the intended use of the modulating agent. For example, microbial AdT protein is used to identify AdT inhibitors that have bactericidal activity whereas chloroplast derived AdT protein is used to identify AdT inhibitors that have herbicide activity.

The method used to identify whether an agent binds to an AdT protein will be based primarily on the nature of the AdT protein used. For example, a gel retardation assay can be used to determine whether an agent binds to a soluble fragment of an AdT protein. Alternatively, immunodetection and biochip technologies can be adopted for use with an AdT protein. A skilled artisan can readily employ numerous art-known techniques for determining whether a particular agent binds to an AdT protein.

Agents can be further tested for the ability to modulate the activity of an AdT protein using a cell-free assay system or a cellular assay system. Example 1 provides one such methods that can be used to assay for AdT activity.

As used herein, an agent is said to antagonize AdT activity when the agent reduces AdT activity. The preferred antagonist will selectively antagonize AdT, not affecting any other cellular proteins, particularly other proteins involved in translation. Further, the preferred antagonist will reduce AdT activity by more than 50%, more preferably by more than 90%, most preferably eliminating all AdT activity.

As used herein, an agent is said to agonize AdT activity when the agent increases AdT activity. The preferred agonist will increase AdT activity by more than 50%, more preferably by more than 90%, most preferably more than doubling the level of AdT activity.

The preferred antagonists and agonists will be selective for a specific species, genus, family, order or kingdom of organisms. Agents can be screened using one AdT protein, or a combination of AdT proteins, to aid in identifying agents for target specificity. For example, several different microbial AdT proteins can be used to identify general antimicrobial agents whereas chloroplast derived AdT proteins can be used to identify herbicide agents.

Agents that are assayed in the above method can be randomly selected or rationally selected or designed. As used herein, an agent is said to be randomly selected when the agent is chosen randomly without considering the specific sequences of the AdT protein. An example of randomly selected agents is the use a chemical library or a peptide combinatorial library, or a growth broth of an organism or plant extract.

As used herein, an agent is said to be rationally selected or designed when the agent is chosen on a nonrandom basis that takes into account the sequence of the target site and/or its conformation in connection with the agent's action. Agents can be rationally selected or rationally designed by utilizing the peptide sequences that make up the AdT protein. For example, a rationally selected peptide agent can be a peptide whose amino acid sequence is identical to a fragment of an AdT protein.

The agents of the present invention can be, as examples, peptides, small molecules, and vitamin derivatives, as well as carbohydrates. A skilled artisan can readily recognize that there is no limit as to the structural nature of the agents of the present invention. One class of agents of the present invention are peptide agents whose amino acid sequences are chosen based on the amino acid sequence of the AdT protein. Small peptide agents can serve as competitive inhibitors of AdT protein assembly.

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene encoded amino acids are to be included.

Another class of agents of the present invention are antibodies immunoreactive with critical positions of the AdT protein. As described above, antibodies are obtained by immunization of suitable mammalian subjects with peptides, containing as antigenic regions, those portions of the AdT protein intended to be targeted by the antibodies. Critical regions include the domains identified in FIG. 2. Such agents can be used in competitive binding studies to identify second generation inhibitory agents.

K. Uses of Agents that Bind to an AdT Protein

As provided in the Background section, the AdT proteins are involved in protein translation, particularly protein translation in gram positive microorganisms, fungi and cellular organelles, particularly chloroplasts. Agents that bind an AdT protein and act as an agonist or antagonist can be used to modulate translation in these organism and serves as a basis for an antibacterial, antifungal or herbicide agents. In detail, protein translation that requires AdT can be modulated by administering to an organism an agent that binds to an AdT protein and acts as an agonist or antagonist of AdT activity.

As used herein, an organism can be any organism, so long as it is desirable to modulate protein translation in the organism, for example to control the growth of an infectious agent in a mammalian subject or to act as an herbicide agent. The invention is particularly useful in the treatment of human subjects for controlling microbial growth.

As used herein, protein translation that requires AdT refers to protein translation that would not occur without the presence of an active AdT protein. As used herein, an agent is said to modulate AdT meditated protein translation when the agent reduces the degree of protein translation.

The use of the AdT modulating agents will be based primarily on the target AdT protein used to identify the agent as well as the activity/selectivity of the agent. For example, an AdT inhibitory agent, that is used as an antimicrobial agent, is preferably isolated using one or more microbial AdT proteins. Herbicide agent will be preferably identified using chloroplast AdT protein as a target.

L. Administration of Agonists and Antagonists of an AdT Protein

The administration of agonists and antagonists of the AdT protein will be dependent on their intended purpose. For example, to control microbial growth in a mammalian subject, an AdT inhibitory agent can be administered via parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired. For example, to treat microbial infection, an agent that modulates AdT activity is administered systemically or locally to the individual being treated. As described below, there are many methods that can readily be adapted to administer such agents.

The present invention further provides compositions containing an antagonist or agonist of an AdT protein that is identified by the methods herein described. The determination of optimal ranges of effective amounts of each component is within the skill of the art and is based on the intended use.

In addition to the AdT modulating agent, the compositions of the present invention may contain other ingredients, such as suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble variant, for example, water-soluble salts. In addition, suspensions of the active compounds and as appropriate, oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension and include, for example, sodium carboxymethyl cellulose, sorbitol, and/or dintran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release variants thereof.

M. Combination Therapy

The agents of the present invention that modulate AdT activity can be provided alone, or in combination with another agents that modulate protein synthesis microbial, fungal or plant growth. For example, an agent of the present invention that reduces microbial AdT activity can be administered in combination with other antimicrobial agents. As used herein, two agents are said to be administered in combination when the two agents are administered simultaneously or are administered independently in a fashion such that the agents will act at the same time.

N. Methods for Identifying the Presence of an AdT Protein or Gene

The present invention further provides methods for identifying cells or organisms expressing an AdT protein or an AdT gene. Such methods can be used to diagnose the presence of an organism that expresses an AdT protein. The methods of the present invention are particularly useful in the determining the presence of pathogenic microorganisms. Specifically, the presence of an AdT protein can be identified by determining whether an AdT protein, or nucleic acid encoding an AdT protein, is expressed. The expression of an AdT protein can be used as a means for diagnosing the presence of an organism that relies on AdT mediated translation.

A variety of immunological and molecular genetic techniques can be used to determine if an AdT protein is expressed/produced in a particular cell or sample. In general, an extract containing nucleic acid molecules or an extract containing proteins is prepared. The extract is then assayed to determine whether an AdT protein, or an AdT encoding nucleic acid molecule, is produced in the cell.

For example, to perform a diagnostic test based on nucleic acid molecules, a suitable nucleic acid sample is obtained and prepared using conventional techniques. DNA can be prepared, for example, simply by boiling a sample in SDS. The extracted nucleic acid can then be subjected to amplification, for example by using the polymerase chain reaction (PCR) according to standard procedures, to selectively amplify an AdT encoding nucleic acid molecule or fragment thereof. The size or presence of a specific amplified fragment (typically following restriction endonuclease digestion) is then determined using gel electrophoresis or the nucleotide sequence of the fragment is determined (for example, see Weber and May Am J Hum Genet (1989) 44:388-339; Davies, J. et al. Nature (1994) 371:130-136)). The resulting size of the fragment or sequence is then compared to the known AdT proteins encoding sequences, for example via hybridization probe. Using this method, the presence of an AdT protein can be identified.

To perform a diagnostic test based on proteins, a suitable protein sample is obtained and prepared using conventional techniques. Protein samples can be prepared, for example, simply by mixing a sample with SDS followed by salt precipitation of a protein fraction. The extracted protein can then be analyzed to determine the presence of an AdT protein using known methods. For example, the presence of specific sized or charged variants of a protein can be identified using mobility in an electric filed. Alternatively, antibodies can be used for detection purposes. A skilled artisan can readily adapt known protein analytical methods to determine if a sample contains an AdT protein.

Alternatively, AdT expression can also be used in methods to identify agents that decrease the level of expression of the AdT gene. For example, cells or tissues expressing an AdT protein can be contacted with a test agent to determine the effects of the agent on AdT expression. Agents that activate AdT expression can be used as an agonist of AdT activity whereas agents that decrease AdT expression can be used as an antagonist of AdT activity.

O. Preparation and Use of Herbicides

As discussed herein, the transamidation pathway is operative in chloroplasts. The ability to identify AdT inhibitors which specifically inhibit plastid isoforms of AdT can be useful in designing herbicides that are not toxic or harmful to humans and animals. Thus, the ability to develop herbicides that inhibit only chloroplast isoforms of enzymes such as Adt but do not inhibit cytosolic (i.e., the fluid portion of the cytoplasm exclusive of organelles) AdT or human AdT, would provide a new form of highly effective herbicide that is also less toxic to humans. However, Adt inhibitors which are not limited to the chloroplasts may also find utility in use as an herbicide.

An identified compound which inhibits function of the wild-type AdT enzyme is utilized as an active ingredient in an herbicide. The active ingredient is normally applied in the form of compositions together with one or more agriculturally acceptable carriers, and can be applied to the crop area or plant to be treated, simultaneously or in succession, with further compounds. These additional compounds can include fertilizers, other herbicides, fungicides, bactericides, nematicides, or mixtures of several of these preparations, together with fisher carriers, surfactants or application-promoting adjuvants. The herbicide may be applied as a seed coating, a ground spray, incorporated into the soil, or applied directly to the plant. Preferably, the active ingredient of the present invention or an agrochemical composition which contains at least one of the active ingredients of the present invention are applied as a leaf preparation. Methods of herbicide preparation and application are well known to one skilled in the art.

Resistant mutants to the AdT-inhibiting compound can be identified by mutagenizing cells or organisms and growing the mutagenized populations in the presence of a concentration of the inhibitor sufficient to inhibit growth of the wild-type cells or organisms, and selecting cells or organisms from the populations that are able to grow more rapidly than wild-type cells or organisms. Mutagenesis can be accomplished by any one of the means well known to one skilled in the art, including: chemical mutatgenesis (e.g., ethyl methanesulfonate); ultraviolet radiation; X-ray exposure; and gamma radiation (see, e.g., Watson et al., Recombinant DNA, Second Edition (1992) Chapter 111:191-211; Freifelder, Molecular Biology (1987) Chapter 11293-313). The mutant individuals which have the ability to tolerate or resist the normally toxic levels of the inhibitor are genetically purified, the gene encoding the mutant AdT is isolated, and the DNA sequence of the mutant gene is determined and translated into a predicted amino acid sequence. The amino acids which differ between the wild-type AdT enzyme and the mutant AdT enzyme are assumed to be responsible for the inhibitor-resistant phenotype of the newly-identified mutant.

The coding DNA sequence for the mutant AdT can be introduced into the plant cell in a number of different ways that are well known to those of skill in the art. Examples of such methods include micro injection, electroporation, Agrobacterium-mediated transformation, direct gene transfer, and micro projectile bombardment. Techniques for producing herbicide resistance in plants by incorporating DNA encoding and expressing enzymes resistant to herbicides are well known (see, e.g., U.S. Pat. No. 5,145,777; U.S. Pat. No. 5,290,926), including techniques for adding a chloroplast transit sequence upstream from an herbicide gene so that the protein product is transported into the cholorplasts (Comai et al., Nature (1985) 313:741-744; U.S. Pat. No. 4,940,835; U.S. Pat. No. 5,188,642). In the same manner, the gene coding for a mutant AdT may be substituted for one of the other herbicide resistance genes of the references. Since AdT performs its function in the chloroplast, it may be particularly relevant to use a plastid transit sequence to ensure expression in the chloroplast or other plastid as is known in the art.

Following introduction of the mutant AdT gene into plant cells and the regeneration of transformed plants from such cells, conventional methods of plant husbandry and plant breeding can be used to maintain and increase the transformed plants. The transformed plants can also be used in conventional hybridization schemes to produce new plant types which also carry the novel mutant AdT gene (see, e.g., Fehr and Hadley, Hybridization of Crop Plants (1980); Jensen, Plant Breeding Methodology (1988); Allard, Principles of Plant Breeding (1960).

Plants which express a gene which is tolerant or resistant to an inhibitor of AdT can be grown in soil and the herbicide containing the AdT inhibitor can be applied to inhibit weed growth. Since the weed plants will not be carrying the mutant AdT gene, the weeds will be susceptible to the herbicide containing the AdT inhibitor.

The following examples are intended to illustrate, but not to limit, aspects of the present invention.

### EXAMPLES

The invention will be flusher described by reference to the following detailed examples. These examples are provided for purposes of illustration only, and are not intended to be limiting unless otherwise specified.

Experimental Procedures

Preparation and purification of recombinant Bacillus subtilis GlntRNA^{Gln} amidotransferase. E. coli BL21 (DE3) harboring pABC were incubated overnight in 3 mL LB medium (10 g bactotryptone, 5 g yeast extract, 10 g NaCl) with 50 (g/mL ampicillin at 37° C. The culture was scaled up to 1 L and again allowed to incubate at 37° C. overnight. Cells were harvested via centrifugation (4000×g for 5 minutes at 4° C.) and resuspended in 20 mL Buffer A (25 mM Hepes.KOH, pH 7.5, 25 mM KCl, 10 mM MgCl2 and 1 mM DTT). This step and all subsequent steps were performed at 4° C. unless otherwise specified. The cells were lysed by sonication (4×15 seconds) and centrifuged at 100,000×g for one hour. The enzyme was then purified to homogeneity, as determined by SDS.polyacrylamide gel electrophoresis, via a series of chromatographic steps using a Pharmacia FPLC system. The supernatant was first applied to a Q-sepharose (HR 16/10) column (strong anion exchange) and the activity was eluted by a linear gradient from 0 to 1 M NaCl in Buffer A. The active fractions from this column were applied to a Superdex-200 (HR 26/100) column (gel filtration) and the activity was eluted isocratically in Buffer A. The fractions from this column which contained activity were pooled and applied onto a MonoQ (HR 10/10) column and the activity as eluted with a linear gradient from 150 to 300 mM NaCl in Buffer A. Active fractions from this column were pooled and dialyzed against Buffer A+200 mM NaCl in 50% glycerol for 12 hours and stored at 70° C.

In vivo expressed Bacillus subtilis tRNA^{Gln} Isolation and purification. A 3 mL culture of E. coli DH5a/pGP1-2/pBTT (encoding tRNAGln) in LB medium (10 g bactotryptone, 5 g yeast extract, 10 g NaCl) with 50 (g/mL ampicillin and 10 (g/mL kanamycin was incubated at 37° C. overnight. The culture was scaled up to 1 L and overnight incubation was repeated. Cells were harvested via centrifugation at 4000×g for 5 minutes at 4° C. and resuspended in 10 mL lysis buffer (20 mm Tris.HCl, pH 7.4 and 20 mM MgCl2). Total nucleic acids were isolated by two sequential extractions with equal volumes of water saturated phenol followed by isopropanol precipitation of the aqueous phase. The nucleic acid pellet was collected via centrifugation at 10,000×g for 15 minutes at 4° C. The pellet was resuspended in 5 mL of 200 mM Tris.OAc, pH 9.0 and incubated at 37° C. for 1 hour to ensure complete deacylation of the tRNA. The nucleic acids were recovered by ethanol precipitation followed by centrifugation at 10,000×g for 15 minutes at 4° C. The pellet was resuspended in 100 mM NaCl, incubated overnight at 4° C. and ethanol precipitated. The tRNAGln was purified by a two-step anion exchange chromatography protocol. The nucleic acids were resuspended in 5 mL of Buffer 1 (140 mM NaOAc, pH 4.5) and 1 gm DE52 resin/100 OD260 was added. The resin was washed with 200 mL Buffer A and 150 mL Buffer 2 (140 mM NaOAc, pH 4.5+300 mM NaCl) and the tRNA was eluted with 100 mL Buffer 3 (140 mM NaOAc, pH 4.5+1 M NaCl). the nucleic acids were recovered by ethanol precipitation followed by centrifugation at 10,000×g for 15 minutes at 4° C. and resuspended in 10 mM Tris.HCl, pH 7.4, 1 mM MgCl2 and 1 mM DTT and applied onto a Pharmacia MonoQ (HR 10/10) column. The tRNA was eluted with a gradient of 450 to 750 mM NaCl in 10 mM Tris.HCl, pH 7.4, 1 mM MgCl2 and 1 mM DTT. Fractions containing the tRNAGln, based on ability to be aminoacylated with both Glu and Gln, were pooled and used as substrates in the amidotransferase assays.

Aminoacylation reactions. The procedure for the formation of radiolabelled GlntRNA^{Gln} was adapted from Jahn, D. et al. (1990). Unless otherwise noted, these reactions were conducted at 37° C. in a buffer consisting of 10 mM ATP, 50 mM Hepes-KOH pH 7.0, 25 mM KCl, 15 mM MgCl2, and 5 mM DTT. The concentration of tRNAGln, recovered from E. coli DH5a harboring the plasmids pGP12 and pBTT (see above), and L14C(U)glutamate (300 mCi/mMol) was 10 (M. GluRS was isolated from B. subtilis and then partially purified by DEAE-sepharose chromatography. The reactions were allowed to progress for various lengths of time depending upon the assay. Aliquots from this mixture were then added to the amidotransferase assay mixtures either directly or following water saturated phenol extraction, ethanol precipitation, and resuspension in the aminoacylation buffer.

Amidotransferase reactions. The procedure for the formation of radiolabelled GlntRNA^{Gln} from GlntRNA^{Gln} was adapted from Jahn, D. et al. (1990). Unless otherwise noted, these reactions were conducted at 37° C. in a buffer consisting of 1 mM ATP, 5 mM Hepes.KOH pH 7.0, 2.5 mM KCl, 1.5 mM MgCl2, and 0.5 mM dithiothreitol (DTT). The concentration of L14C(U)-GlutRNA^{Gln} was 1 (M and the concentration of L glutamine was 1 mM. Aliquots (0 to 20 (L) from fractions obtained during purification of the enzyme were added and the mixture was incubated for various lengths of time depending upon the assay followed by quenching with 10 (L, 3 M NaOAc, pH 5.0. The mixture was extracted with an equal volume of water-saturated phenol and the aqueous and organic phases were separated by centrifugation at 15,000×g at room temperature for 60 seconds. The aqueous phase was removed, 3× volumes of ethanol were added and the tRNA was precipitated at 70° C. for 15 minutes. The precipitated tRNA was recovered by centrifugation at 15,000×g at 4° C. for 15 minutes. The pellet obtained was resuspended in 50 (L 0.01 N KOH and deaminoacylated at 65° C. for 10 minutes. The base was neutralized with 1.3 (L, 0.1 N HCl (to pH (6 to 7) and the solution was dried completely under vacuum. The dried pellet was resuspended in 3 (L double-distilled H₂O and spotted onto a TLC place (cellulose, Aldrich). The front was allowed to migrate 3.5 to 5 hours in one of two solvent systems (A. 20:1:5 isopropyl alcohol:formic acid:water or B. 2:1:6:6 ammonia:water:chloroform:methanol). The plate was dried at 85° C., exposed to an activated phosphoroimaging plate ((12 hours) and the image was analyzed using MacBas v2.0. In this way, the conversion of Glu to Gin was measured.

### Example 1

Characterization of DNA Fragment Encoding AdT

Three genes; one transcript of correct size hybridizing with three probes is provided in FIG. 3 (SEQ ID NO:1). Open reading frames for each of the subunits is provided.

### Example 2

Characterization of B. subtilis AdT Protein

The amino acid sequence of B. subtilis AdT is encoded by the nucleotide sequence of FIG. 3 (SEQ ID NO:1).

The molecular weights of the three subunits is computed to be: 53.039 Kd, A subunit; 53.314 Kd, B subunit; and 10.859 Kd, C subunit. The amino acid sequences of each of the subunits A, B and C are provided in SEQ ID NOs: 4, 6 and 8, respectively.

The sizes of the subunits were confirmed via polyacrylamide gel electrophoresis.

### Example 3

Preparation of Polyclonal Antiserum Containing Anti-AdT Antibodies

A polyclonal antiserum containing anti-AdT antibodies was obtained by administered to rabbits recombinant AdT (trimeric protein) to rabbits following known methods. Incubation of the antiserum with a B. subtilis extract containing AdT protein completely inhibited AdT activity.

Using polyacrylamide gel electrophoresis, the antiserum was shown to contain antibodies immunoreactive to the assembled AdT protein. The polyclonal antisera was significantly less immunoreactive to non-assembled, individual subunits.

### Example 4

Production and Purification of AdT

TABLE I
Activity of cell extracts from E. coli BL21(DE3)
harboring various vectors.

| | Glutamine Recovered | |
|---|---|---|
| Vector | pMole | Relative Activity |
| pABC | 2.03 ± 0.28 | 1.000 |
| pA | 0.02 ± 0.02 | 0.010 |
| pB | 0.03 ± 0.01 | 0.015 |
| pC | 0.03 ± 0.02 | 0.017 |

TABLE II
Purification of Bacillus subtilis GlntRNAGln amidotransferase
over expressed in Escherichia coli BL21(DE3).

| Purification | Total | Total | Total | Specific | | |
|---|---|---|---|---|---|---|
| Step | volume | protein | activity^{a} | activity | Yield | |
| Purification | mL | mg | units × 10³ | units/mg | % | -fold |
| S-100 | 15 | 190 | 13.5 | 70 | 100 | 1 |
| Q · | 12 | 40 | 8.1 | 200 | 60 | 3 |
| sepharoseFF | | | | | | |
| Superdex- | 10 | 4.6 | 13.4 | 3000 | 99 | 40 |
| 200 | | | | | | |
| MonoQ | 3.5 | 1.0 | 8.3 | 9000 | 61 | 130 |

^{a}One unit is defined as 1 pMole glutamine produced per minute at 37° C. under the assay conditions described in materials and methods.

### Example 5

Identifying Inhibitors of AdT Activity

Purified amidotransferase is used in an assay to identify inhibitors of AdT activity. The assay used to identify inhibitors of AdT activity comprises:
(a) incubating a first sample of AdT and its substrate;
(b) measuring an uninhibited reactivity of the AdT from step (a);
(c) incubating a first sample of AdT and its substrate in the presence of a second sample comprising an inhibitor compound;
(d) measuring an inhibited reactivity of the AdT from step (c); and,
(e) comparing the inhibited reactivity to the uninhibited reactivity of the AdT.

Inhibitors of AdT identified using this process are utilized as antibacterial, antifungal and herbicidal agents.

### Example 6

Identification of Inhibitor-Resistant AdT Mutants

Purified amidotransferase and an identified inhibitor of AdT is used in an assay to identify inhibitor-resistant AdT mutants. The assay used to identify inhibitor-resistant AdT mutants comprises:
(a) incubating a first sample of AdT and its substrate in the presence of a second sample comprising an AdT inhibitor;
(b) measuring an unmutated reactivity of the AdT from step (a);
(c) incubating a first sample of a mutated AdT and its substrate in the presence of a second sample comprising an AdT inhibitor,
(d) measuring a mutated reactivity of the mutated AdT from step (c); and,
(e) comparing the mutated reactivity to the unmutated reactivity of the AdT.

Inhibitor-resistant AdT mutants identified using this process are utilized in the production of cells and organisms resistant to AdT inhibitors.

### Example 7

Diagnostic Assays

Nucleic acids for diagnosis are obtained from cells or tissues. Genomic DNA may be used directly for detection or may be amplified enzymatically by using PCR or other amplification techniques prior to analysis. The genomic DNA can be compared to the polynucleotide coding for amidotransferase as provided in SEQ ID NO: 1. Deletions and insertions can be detected by a change in size of the amplified product in comparison to SEQ ID NO:1. Point mutations can be identified by hybridizing amplified DNA to labeled AdT polynucleotide sequences. Perfectly matched sequences can be distinguished from mismatched duplexes by RNASE digestion or by differences in melting temperatures. DNA sequence differences may also be detected by alterations in the electrophoretic mobility of the DNA fragments in gels, with or without denaturing agents, or by direct DNA sequencing. Sequence changes at specific locations also may be revealed by nuclease protection assays, such as RNase and S1 protection or a chemical cleavage method.

Cells carrying mutations or polymorphisms in the gene of the invention may also be detected at the DNA level by a variety of techniques. For example, RTPCR can be used to detect mutations. It is particularly preferred to used RTPCR in conduction with automated detection systems, such as, for example, GeneScan. RNA or cDNA may also be used for the same purpose, PCR or RTPCR. As an example, PCR primers complementary to the nucleic acid encoding AdT can be used to identify and analyze mutations. These primers may be used for amplifying AdT DNA isolated from a sample derived from an organism. The invention also provides these primers with 1, 2, 3 or 4 nucleotides removed from the 5′ and/or the 3′ end. The primers may be used to amplify the gene isolated from an infected individual such that the gene may then be subject to various techniques for elucidation of the DNA sequence. In this way, mutations in the AdT DNA sequence may be detected and used to diagnose infection and to serotype or classify the infectious agent.

Increased or decreased expression of AdT polynucleotide can be measured using any of the methods well known in the art for the quantitation of polynucleotides, such as, for example, amplification, PCR, RTPCR, RNase protection, Northern blotting and other hybridization methods.

In addition, a diagnostic assay in accordance with the intention for detecting over-or under-expression of AdT protein compared to normal control tissue samples. Assay techniques that can be sued to determine levels of AdT protein, in a sample derived from a host are well-known to those of skill in the art. Such assay methods include radioimmunoassay, competitive-binding assays, Western Blot analysis and ELISA assays.

### Example 8

Production of Transformed Plants

A mutated AdT-encoding DNA sequence Fat confers resistance to AdT inhibitors is isolated from an inhibitor-resistant AdT mutant using mutagenesis and isolation techniques well known to one of skill in the art. The coding sequence for the mutant AdT gene is then introduced into a plant cell and whole transformed plants are regenerated from the transformed plant cell using a number of different techniques well known to those of skill in the art. The transformed plants are used in conventional plant breeding schemes to produce new varieties of plants which also carry the mutant AdT gene. Crop plants carrying the mutant AdT gene are grown in production and an herbicide comprising an AdT inhibitor is applied to the crop to control weeds.

Although the present invention has been described in detail with reference to examples above, it is understood that various modifications can be made without departing from the spirit of the invention. All cited references referred to in the application are hereby incorporated by reference.

1. An isolated polynucleotide encoding a GlutRNA^{Gln} amidotransferase (AdT) protein.

2. An isolated polynucleotide comprising a polynucleotide sequence selected from the group consisting of:
(a) a polynucleotide having at least a 70% identity to a polynucleotide encoding a polypeptide encoded by SEQ ID NO:1; and
(b) a polynucleotide which is complementary to the polynucleotide of (a).

3. The polynucleotide of claim 2 wherein the polynucleotide is DNA.

4. The polynucleotide of claim 2 wherein the polynucleotide is RNA.

5. The polynucleotide of claim 3 comprising the nucleotides 103 to 3306 set forth in SEQ ID NO:1.

6. A vector comprising the DNA of claim 3.

7. A host cell comprising the vector of claim 6.

8. A process for producing a polypeptide comprising expressing from the host cell of claim 7 a polypeptide encoded by said DNA.

9. A process for producing a cell which expresses a polypeptide comprising transforming or transfecting the cell with the vector of claim 6 such that the cell expresses the polypeptide encoded by the DNA contained in the vector.

10. A process for producing an amidotransferase polypeptide or amidotransferase fragment comprising culturing a host cell of claim 7 under conditions sufficient for the production of said polypeptide or fragment.

11. A polypeptide comprising an amino acid sequence which is at least 70% identical to a polypeptide encoded by nucleotides 103 to 3306 set forth in SEQ ID NO:1.

12. An antibody, or antibody fragment containing an antigen binding site, wherein said antibody binds to a polypeptide of claim 11.

13. An antagonist which inhibits the activity of the polypeptide of claim 11.

14. A method for the treatment of an individual having need of amidotransferase comprising administering to the individual a therapeutically effective amount of the polypeptide of claim 11.

15. The method of claim 14 wherein said therapeutically effective amount of the polypeptide is administered by providing to the individual DNA encoding said polypeptide and expressing said polypeptide in vivo.

16. A method for the treatment of an individual having need to inhibit amidotransferase polypeptide comprising administering to the individual a therapeutically effective amount of the antagonist of claim 13.

17. A process for diagnosing a disease related to expression of the polypeptide of claim 11 comprising determining a nucleic acid sequence encoding said polypeptide.

18. A diagnostic process comprising analyzing for the presence of the polypeptide of claim 11 in a sample derived from a host.

19. A method for identifying compounds which bind to and inhibit an activity of a polypeptide of claim 11 comprising:
(a) incubating a first sample of the polypeptide and its substrate;
(b) measuring an uninhibited reactivity of the polypeptide from step (a);
(c) incubating a first sample of the polypeptide and its substrate in the presence of a second sample comprising an inhibitor compound;
(d) measuring an inhibited reactivity of the polypeptide from step (c); and
(e) comparing the inhibited reactivity to the uninhibited reactivity of the polypeptide.

20. A method for identifying compounds which bind to and inhibit an activity of a polypeptide of claim 11 comprising:
(a) contacting a cell expressing on the surface thereof a binding site for the polypeptide, said binding being associated with a second component capable of providing a detectable signal in response to the binding of a compound to said binding site, with a compound to be screened under conditions to permit binding to the binding site; and
(b) determining whether the compound binds to and activates or inhibits the binding by detecting the presence or absence of a signal generated from the interaction of the compound with the binding site.

21. A method for identifying inhibitor-resistant AdT mutants comprising:
(a) incubating a first sample of wild-type AdT and its substrate in the presence of a second sample comprising an AdT inhibitor;
(b) measuring an unmutated reactivity of the AdT from step (a);
(c) incubating a first sample of a mutated AdT and its substrate in the presence of a second sample comprising an AdT inhibitor;
(d) measuring a mutated reactivity of the mutated AdT from step (c); and,
(e) comparing the mutated reactivity to the unmutated reactivity of the wild-type AdT.

22. A method for inducing an immunological response in a mammal which comprises inoculating the mammal with amidotransferase, or a fragment or variant thereof adequate to produce antibody to protect said animal from disease.

23. A method of inducing immunological response in a mammal which comprises, through gene therapy, delivering a gene encoding an amidotransferase fragment or a variant thereof for expressing amidotransferase, or a fragment or a variant thereof in vivo in order to induce an immunological response to produce antibody to protect said animal from disease.

24. An immunological composition comprising a DNA which codes for and expresses an amidotransferase polynucleotide or protein coded therefrom which, when introduced into a mammal, induces an immunological response in the mammal to a given amidotransferase polynucleotide or protein coded therefrom.

25. An isolated polynucleotide comprising a polynucleotide sequence selected from the group consisting of:
(a) a polynucleotide having at least a 70% identity to a polynucleotide encoding a polypeptide comprising SEQ ID NO:4; and
(b) a polynucleotide which is complementary to the polynucleotide of (a).

26. A vector comprising the polynucleotide of claim 25.

27. A host cell comprising the vector of claim 26.

28. A process for producing a polypeptide comprising expressing from the host cell of claim 27 a polypeptide encoded by said polynucleotide.

29. A polypeptide comprising an amino acid sequence which is at least 70% identical to the amino acid set forth in SEQ ID NO:4.

30. An antibody, or antibody fragment containing an antigen binding site, wherein said antibody binds to a polypeptide of claim 29.

31. An antagonist which inhibits the activity of the polypeptide of claim 29.

32. An isolated polynucleotide comprising a polynucleotide sequence selected from the group consisting of:
(a) a polynucleotide having at least a 70% identity to a polynucleotide encoding a polypeptide comprising SEQ ID NO:6; and
(b) a polynucleotide which is complementary to the polynucleotide of (a).

33. A vector comprising the polynucleotide of claim 32.

34. A host cell comprising the vector of claim 33.

35. A process for producing a polypeptide comprising expressing from the host cell of claim 34 a polypeptide encoded by said polynucleotide.

36. A polypeptide comprising an amino acid sequence which is at least 70% identical to the amino acid set forth in SEQ ID NO:6.

37. An antibody, or antibody fragment containing an antigen binding site, wherein said antibody binds to a polypeptide of claim 36.

38. An antagonist which inhibits the activity of the polypeptide of claim 36.

39. An isolated polynucleotide comprising a polynucleotide sequence selected from the group consisting of:
(a) a polynucleotide having at least a 70% identity to a polynucleotide encoding a polypeptide comprising SEQ ID NO:8; and
(b) a polynucleotide which is complementary to the polynucleotide of (a).

40. A vector comprising the polynucleotide of claim 39.

41. A host cell comprising the vector of claim 40.

42. A process for producing a polypeptide comprising expressing from the host cell of claim 41 a polypeptide encoded by said polynucleotide.

43. A polypeptide comprising an amino acid sequence which is at least 70% identical to the amino acid set forth in SEQ ID NO:8.

44. An antibody, or antibody fragment containing an antigen binding site, wherein said antibody binds to a polypeptide of claim 43.

45. An antagonist which inhibits the activity of the polypeptide of claim 43.

46. An isolated heterotrimeric protein comprising subunits A, B, and C, wherein:
said subunit A has an amino acid sequence selected from the group consisting of SEQ ID NO:4, an allelic variant of SEQ ID NO:4, a conservative substitution variant of SEQ ID NO:4, and an amino acid sequence that is encoded by a nucleic acid molecule that hybridizes under stringent conditions to a nucleic acid molecule encoding SEQ ID NO:4 and encodes a subunit of a heterotrimeric amidotransferase;
said subunit B has an amino acid sequence selected from the group consisting of SEQ ID NO:6, an allelic variant of SEQ ID NO:6, a conservative substitution variant of SEQ ID NO:6, and an amino acid sequence that is encoded by a nucleic acid molecule that hybridizes under stringent conditions to a nucleic acid molecule encoding SEQ ID NO:6 and encodes a subunit of a heterotrimeric amidotransferase;
said subunit C has an amino acid sequence selected from the group consisting of SEQ ID NO:8, an allelic variant of SEQ ID NO:8, a conservative substitution variant of SEQ ID NO:8, and an amino acid sequence that is encoded by a nucleic acid molecule that hybridizes under stringent conditions to a nucleic acid molecule encoding SEQ ID NO:8 and encodes a subunit of a heterotrimeric amidotransferase.

47. An antibody, or antibody fragment containing an antigen binding site, wherein said antibody binds to a protein of claim 46.

48. An isolated nucleic acid molecule that encodes a protein of claim 46.

49. A recombinant host that has been altered to contain a nucleic acid molecule of claim 48.

50. A method for producing an AdT protein comprising the step of culturing the host of claim 49 under conditions in which said introduced nucleic acid molecule is expressed.

51. A method to identify an agent that blocks translation, said method comprising the steps of:
(a) contacting an agent with an AdT protein, or a subunit thereof; and,
(b) determining whether said agent binds to said AdT protein or said subunit;
wherein said translation blocking agent is identified as being able to bind to said AdT protein, or said subunit.

52. The method of claim 51, wherein said AdT protein comprises a heterotrimeric protein consisting of an A, B and C subunit, wherein:
said subunit A has an amino acid sequence selected from the group consisting of SEQ ID NO:4, an allelic variant of SEQ ID NO:4, a conservative substitution variant of SEQ ID NO:4, and an amino acid sequence that is encoded by a nucleic acid molecule that hybridizes under stringent conditions to a nucleic acid molecule encoding SEQ ID NO:4 and encodes a subunit of a heterotrimeric amidotransferase;
said subunit B has an amino acid sequence selected from the group consisting of SEQ ID NO:6, an allelic variant of SEQ ID NO:6, a conservative substitution variant of SEQ ID NO:6, and an amino acid sequence that is encoded by a nucleic acid molecule that hybridizes under stringent conditions to a nucleic acid molecule encoding SEQ ID NO:6 and encodes a subunit of a heterotrimeric amidotransferase;
said subunit C has an amino acid sequence selected from the group consisting of SEQ ID NO:8, an allelic variant of SEQ ID NO:8, a conservative substitution variant of SEQ ID NO:8, and an amino acid sequence that is encoded by a nucleic acid molecule that hybridizes under stringent conditions to a nucleic acid molecule encoding SEQ ID NO:8 and encodes a subunit of a heterotrimeric amidotransferase.

53. The method of claim 52, wherein a single subunit of said AdT protein is used, and:
if subunit A is used, said subunit A has an amino acid sequence selected from the group consisting of SEQ ID NO:4, an allelic variant of SEQ ID NO:4, a conservative substitution variant of SEQ ID NO:4, and an amino acid sequence that is encoded by a nucleic acid molecule that hybridizes under stringent conditions to a nucleic acid molecule encoding SEQ ID NO:4 and encodes a subunit of a heterotrimeric amidotransferase;
if subunit B is used, said subunit B has an amino acid sequence selected from the group consisting of SEQ ID NO:6, an allelic variant of SEQ ID NO:6, a conservative substitution variant of SEQ ID NO:6, and an amino acid sequence that is encoded by a nucleic acid molecule that hybridizes under stringent conditions to a nucleic acid molecule encoding SEQ ID NO:6 and encodes a subunit of a heterotrimeric amidotransferase;
if subunit C is used, said subunit C has an amino acid sequence selected from the group consisting of SEQ ID NO:8, an allelic variant of SEQ ID NO:8, a conservative substitution variant of SEQ ID NO:8, and an amino acid sequence that is encoded by a nucleic acid molecule that hybridizes under stringent conditions to a nucleic acid molecule encoding SEQ ID NO:8 and encodes a subunit of a heterotrimeric amidotransferase.

54. The method of claim 51, wherein said agent is further tested for the ability to block the activity of said AdT protein.

55. The method of claim 54, wherein said AdT activity is tested in a cell free system.

56. The method of claim 54, wherein said AdT activity is tested in a cellular system.

57. A method to identify an agent that blocks translation, said method comprising the steps of:
(a) contacting an agent with one or more of the subunits of an AdT protein;
(b) incubating the three subunits of an AdT protein under conditions in which said subunits would associate to form an active AdT protein, wherein at least one of said subunits is from step (a); and
(c) determining whether said agent blocks the association of said three subunits;
wherein said translation blocking agent is identified as being able to block the association of the subunits of said AdT protein.

58. The method of claim 57, wherein said AdT protein comprises a heterotrimeric protein consisting of an A, B and C subunit, wherein:
said subunit A has an amino acid sequence selected from the group consisting of SEQ ID NO:4, an allelic variant of SEQ ID NO:4, a conservative substitution variant of SEQ ID NO:4, and an amino acid sequence that is encoded by a nucleic acid molecule that hybridizes under stringent conditions to a nucleic acid molecule encoding SEQ ID NO:4 and encodes a subunit of a heterotrimeric amidotransferase;
said subunit B has an amino acid sequence selected from the group consisting of SEQ ID NO:6, an allelic variant of SEQ ID NO:6, a conservative substitution variant of SEQ ID NO:6, and an amino acid sequence that is encoded by a nucleic acid molecule that hybridizes under stringent conditions to a nucleic acid molecule encoding SEQ ID NO:6 and encodes a subunit of a heterotrimeric amidotransferase;
said subunit C has an amino acid sequence selected from the group consisting of SEQ ID NO:8, an allelic variant of SEQ ID NO:8, a conservative substitution variant of SEQ ID NO:8, and an amino acid sequence that is encoded by a nucleic acid molecule that hybridizes under stringent conditions to a nucleic acid molecule encoding SEQ ID NO:8 and encodes a subunit of a heterotrimeric amidotransferase.

59. The method of claim 57, wherein said agent is further tested for the ability to block the activity of said AdT protein.

60. The method of claim 59, wherein said AdT protein activity is tested in a cell free system.

61. The method of claim 59, wherein said AdT protein activity is tested in a cellular system.

62. A method to identify an agent that blocks translation, said method comprising the steps of:
(a) contacting an agent with an AdT protein;
(b) determining whether said agent blocks the activity of said AdT protein.

63. The method of claim 62, wherein said AdT protein comprises a heterotrimeric protein consisting of an A, B and C subunit, wherein:
said subunit A has an amino acid sequence selected from the group consisting of SEQ ID NO:4, an allelic variant of SEQ ID NO:4, a conservative substitution variant of SEQ ID NO:4, and an amino acid sequence that is encoded by a nucleic acid molecule that hybridizes under stringent conditions to a nucleic acid molecule encoding SEQ ID NO:4 and encodes a subunit of a heterotrimeric amidotransferase;
said subunit B has an amino acid sequence selected from the group consisting of SEQ ID NO:6, an allelic variant of SEQ ID NO:6, a conservative substitution variant of SEQ ID NO:6, and an amino acid sequence that is encoded by a nucleic acid molecule that hybridizes under stringent conditions to a nucleic acid molecule encoding SEQ ID NO:6 and encodes a subunit of a heterotrimeric amidotransferase;
said subunit C has an amino acid sequence selected from the group consisting of SEQ ID NO:8, an allelic variant of SEQ ID NO:8, a conservative substitution variant of SEQ ID NO:8, and an amino acid sequence that is encoded by a nucleic acid molecule that hybridizes under stringent conditions to a nucleic acid molecule encoding SEQ ID NO:8 and encodes a subunit of a heterotrimeric amidotransferase.

64. The method of claim 63, wherein said AdT protein activity is tested in a cell free system.

65. The method of claim 63, wherein said AdT protein activity is tested in a cellular system.

66. A method to block translation of a protein within a cell, comprising the step of contacting said cell with an amount of an agent that binds to an AdT protein, or a subunit thereof, sufficient to block said translation.

67. The method of claim 66, wherein said agent binds to a subunit of said AdT and blocks the association of said subunits.

68. The method of claim 66, wherein said agent is used as an antibacterial agent.

69. The method of claim 66, wherein said agent is used as an antifungal agent.

70. The method of claim 66, wherein said agent is used as a herbicide.

71. An isolated polynucleotide that codes for a mutant AdT which confers resistance to an inhibitor of wild-type AdT.

72. A vector comprising the polynucleotide of claim 71.

73. A host cell comprising the vector of claim 72.

74. The host cell of claim 73 wherein the host cell comprises a plant cell.

75. A process for producing a polypeptide comprising expressing from the host cell of claim 73 a polypeptide encoded by said polynucleotide.

76. A process for producing a cell which expresses a polypeptide comprising transforming or transfecting the cell with the vector of claim 73 such that the cell expresses the polypeptide encoded by the polynucleotide contained in the vector.

77. A process for producing a plant which comprises a gene for resistance to an AdT inhibitor, said process comprising regenerating a plant from the plant cell of claim 74.

78. A process of plant husbandry comprising:
(a) planting a plant which comprises a gene for resistance to an AdT inhibitor; and
(b) applying a herbicide which comprises an AdT inhibitor.


SEQUENCE LISTING (8 sequences)

SEQ ID NO: 1 - DNA, 3495 bp, Bacillus subtilis
  misc_feature (2330)..(2330): n is a, c, g, or t
gaattcgatcctgtctcaaggcgttttgttgctttaaagggcttgtttttgatatgatcagtattatatgacttaacggagaaatatgtggaggtggatcatatgtcacgaatttcaatagaagaagtaaagcacgttgcgcaccttgcaagacttgcgattactgaagaagaagcaaaaatgttcactgaacagctcgacagtatcatttcatttgccgaggagcttaatgaggttaacacagacaatgtggagcctacaactcacgtgctgaaaatgaaaaatgtcatgagagaagatgaagcgggtaaaggtcttccggttgaggatgtcatgaaaaatgcgcctgaccataaagacggctatattcgtgtgccatcaattctggactaaaggagggacacaagaatgtcattatttgatcataaaatcacagaattaaaacagctcatacataaaaaagagattaagatttctgatctggttgatgaatcttataaacgcatccaagcggttgatgataaggtacaagcctttttggcattagatgaagaaagagcgcgcgcatacgcgaaggagcttgatgaggcggttgacggccgttctgagcacggtcttcttttcggtatgcccatcggcgtaaaagataatatcgtaacaaaagggctgcgcacaacatgctccagcaaaattctcgaaaactttgatccgatttacgatgctactgtcgttcagcgccttcaagacgctgaagcggtcacaatcggaaaactgaacatggacgaattcgccatggggtcatctacagaaaactcagcttacaagctgacgaaaaacccttggaacctggatacagttcccggcggttcaagcggcggatctgcagctgcggttgctgcgggagaagttccgttttctcttggatctgacacaggcggctccatccgtcagccggcatctttctgcggcgttgtcggattaaaacctacatacggacgtgtatctcgttacggcctggtcgcatttgcgtcttcattggaccaaatcggaccgattacacgtacggttgaggataacgcgtttttacttcaagcgatttccggcgtagacaaaatggactctacgagtgcaaatgtggacgtgcctgattttctttcttcattaactggcgacatcaaaggactgaaaatcgccgttccgaaagaataccttggtgaaggtgtcggcaaagaagcgagagaatctgtcttggcagcgctgaaagtccttgaaggtctcggcgctacatgggaagaagtgtctcttccgcacagtaaatacgcgcttgcgacatattacctgctgtcatcttctgaagcgtcagcgaaccttgcacgctttgacggcatccgctacggctaccgcacagacaacgcggataacctgatcgacctttacaagcaaacgcgcgctgaaggtttcggaaatgaagtcaaacgccgcatcatgctcggaacgtttgctttaagctcaggctactacgatgcgtactacaaaaaagcgcaaaaagtgcgtacgttgattaagaaggatttcgaggacgtatttgaaaaatatgatgttattgttggaccgactacaccgacacctgcgtttaaaatcggtgaaaacacgaaggatccgctcacaatgtacgcaaacgatatcttaacgattccggtcaaccttgcggcgtaccgggaatcaggtgccatgcggttagcagacggacttccgctcggcctgcaaatcatcggaaaacactttgatgaagcactgtataccgcgttgctcatgcatttgaacaagcaacagaccatcataaagcaaaacctgaactgtaaggggtgaaaagaattgaactttgaaacggtaatcggacttgaagtccacgttgagttaaaaacaaaatcaaaaattttctcaagctctccaacgccattcggcgcggaggcgaatacgcagacaagcgttattgacctcggatatccgggcgtcctgcctgttctgaacaaagaagccgttgaattcgcaatgaaagccgctatggcgctcaactgtgagatcgcaacggatacgaagtttgaccgcaaaaactatttctatcctgacaacccgaaagcgtatcagatttctcaatttgataagccaatcggcgaaaacggctggatcgaaattgaagtcggcggcaaaacaaaacgcatcggcatcacgcgccttcatcttgaagaggatgccggaaaactgacgcatacgggcgacggctattctcttgttgacttcaaccgtcaaggaacgccgcttgttgagtncgtatcagagccggacatccgcacgccggaagaancgtacgcatatcttgaaaagctgaaatccatcatccaatatacaggcgtttctgactgtaaaatggaagaaggctcacttcgctgtgacgccaatatctctcttcgtccgatcggccaagaggaattcggcacaaaaacagaattgaaaaacttgaactcctttgcgtttgttcaaaaaggccttgagcatgaagaaaaacgccaggagcaggttcttctttccggcttcttcatccagcaagaaactcgccgttatgatgaagcaacgaagaaaaccattcttatgcgtgtcaaagagggatctgacgactaccgttactttccagagccagatctagtcgagctctacattgatgatgaatggaaggaacgcgtaaaagcaagcattcctgagcttccggatgagcgccgcaagcgttatatcgaagagcttggcttcgctgcatatgacgcaatggttctgacgctgacaaaagaaatggctgatttcttcgaagaaaccgttcaaaaaggcgctgaagctaaacaagcgtctaactggctgatgggtgaagtgtcagcttacctaaacgcagaacaaaaagagcttgccgatgttgccctgacacctgaaggccttgccggcatgatcaaattgattgaaaaaggaaccatttcttctaagatcgcgaagaaagtgtttaaagaattgattgaaaaaggcggcgacgctgagaagattgtgaaagagaaaggccttgttcagatttctgacgaaggcgtgcttctgaagcttgtcactgaggcgcttgacaacaatcctcaatcaatcgaagactttaaaaacggaaaagaccgcgcgatcggcttcctagtcggacagattatgaaagcgtccaaaggacaagccaacccgccgatggtcaacaaaattctgcttgaagaaattaaaaaacgctaataaaaaagcagcccttagaggctgctttttttatggtcaaattgagataaagacaagatgagggcccgaagcctttcaacttctttgtcgttggttccggccaaattggacagcatgcctttataatcggcttgcgcggtttatcctgagtcaattcttcctcgataagataagtgacacggtgatatc

SEQ ID NO: 2 - protein, 1155 aa, Bacillus subtilis
  misc_feature (774)..(774): Xaa is Phe, Ser, Tyr, or Cys
EFDPVSRRFVALKGLFLISVLYDLTEKYVEVDHMSRISIEEVKHVAHLARLAITEEEAKMFTEQLDSIISFAEELNEVNTDNVEPTTHVLKMKNVMREDEAGKGLPVEDVMKNAPDHKDGYIRVPSILDRRDTRMSLFDHKITELKQLIHKKEIKISDLVDESYKRIQAVDDKVQAFLALDEERARAYAKELDEAVDGRSEHGLLFGMPIGVKDNIVTKGLRTTCSSKILENFDPIYDATVVQRLQDAEAVTIGKLNMDEFAMGSSTENSAYKLTKNPWNLDTVPGGSSGGSAAAVAAGEVPFSLGSDTGGSIRQPASFCGVVGLKPTYGRVSRYGLVAFASSLDQIGPITRTVEDNAFLLQAISGVDKMDSTSANVDVPDFLSSLTGDIKGLKIAVPKEYLGEGVGKEARESVLAALKVLEGLGATWEEVSLPHSKYALATYYLLSSSEASANLARFDGIRYGYRTDNADNLIDLYKQTRAEGFGNEVKRRIMLGTFALSSGYYDAYYKKAQKVRTLIKKDFEDVFEKYDVIVGPTTPTPAFKIGENTKDPLTMYANDILTIPVNLAAYRESGAMRLADGLPLGLQIIGKHFDEALYTALLMHLNKQQTIIKQNLNCKGKELNFETVIGLEVHVELKTKSKIFSSSPTPFGAEANTQTSVIDLGYPGVLPVLNKEAVEFAMKAAMALNCEIATDTKFDRKNYFYPDNPKAYQISQFDKPIGENGWIEIEVGGKTKRIGITRLHLEEDAGKLTHTGDGYSLVDFNRQGTPLVEXVSEPDIRTPEEXYAYLEKLKSIIQYTGVSDCKMEEGSLRCDANISLRPIGQEEFGTKTELKNLNSFAFVQKGLEHEEKRQEQVLLSGFFIQQETRRYDEATKKTILMRVKEGSDDYRYFPEPDLVELYIDDEWKERVKASIPELPDERRKRYIEELGFAAYDAMVLTLTKEMADFFEETVQKGAEAKQASNWLMGEVSAYLNAEQKELADVALTPEGLAGMIKLIEKGTISSKIAKKVFKELIEKGGDAEKIVKEKGLVQISDEGVLLKLVTEALDNNPQSIEDFKNGKDRAIGFLVGQIMKASKGQANPPMVNKILLEEIKKRKSSPRLLFLWSNDKDKMRARSLSTSLSLVPAKLDSMPLSACAVYPESILPRDKHGDI

SEQ ID NO: 3 - DNA, 1461 bp, Bacillus subtilis
  CDS (1)..(1458): Subunit A, corresponds to nucleotides 409-1869 of SEQ ID NO 1
atgtcattatttgatcataaaatcacagaattaaaacagctcatacataaaaaagagattaagatttctgatctggttgatgaatcttataaacgcatccaagcggttgatgataaggtacaagcctttttggcattagatgaagaaagagcgcgcgcatacgcgaaggagcttgatgaggcggttgacggccgttctgagcacggtcttcttttcggtatgccgatcggcgtaaaagataatatcgtaacaaaagggctgcgcacaacatgctccagcaaaattctcgaaaactttgatccgatttacgatgctactgtcgttcagcgccttcaagacgctgaagcggtcacaatcggaaaactgaacatggacgaattcgccatggggtcatctacagaaaactcagcttacaagctgacgaaaaacccttggaacctggatacagttcccggcggttcaagcggcggatctgcagctgcggttgctgcgggagaagttccgttttctcttggatctgacacaggcggctccatccgtcagccggcatctttctgcggcgttgtcggattaaaacctacatacggacgtgtatctcgttacggcctggtcgcatttgcgtcttcattggaccaaatcggaccgattacacgtacggttgaggataacgcgtttttacttcaagcgatttccggcgtagacaaaatggactctacgagtgcaaatgtggacgtgcctgattttctttcttcattaactggcgacatcaaaggactgaaaatcgccgttccgaaagaataccttggtgaaggtgtcggcaaagaagcgagagaatctgtcttggcagcgctgaaagtccttgaaggtctcggcgctacatgggaagaagtgtctcttccgcacagtaaatacgcgcttgcgacatattacctgctgtcatcttctgaagcgtcagcgaaccttgcacgctttgacggcatccgctacggctaccgcacagacaacgcggataacctgatcgacctttacaagcaaacgcgcgctgaaggtttcggaaatgaagtcaaacgccgcatcatgctcggaacgtttgctttaagctcaggctactacgatgcgtactacaaaaaagcgcaaaaagtgcgtacgttgattaagaaggatttcgaggacgtatttgaaaaatatgatgttattgttggaccgactacaccgacacctgcgtttaaaatcggtgaaaacacgaaggatccgctcacaatgtacgcaaacgatatcttaacgattccggtcaaccttgcggcgtaccgggaatcaggtgccatgcggttagcagacggacttccgctcggcctgcaaatcatcggaaaacactttgatgaagcactgtataccgcgttgctcatgcatttgaacaagcaagagaccatcataaagcaaaacctgaactgtaaggggtga

SEQ ID NO: 4 - protein, 486 aa, Bacillus subtilis
MSLFDHKITELKQLIHKKEIKISDLVDESYKRIQAVDDKVQAFLALDEERARAYAKELDEAVDGRSEHGLLFGMPIGVKDNIVTKGLRTTCSSKILENFDPIYDATVVQRLQDAEAVTIGKLNMDEFAMGSSTENSAYKLTKNPWNLDTVPGGSSGGSAAAVAAGEVPFSLGSDTGGSIRQPASFCGVVGLKPTYGRVSRYGLVAFASSLDQIGPITRTVEDNAFLLQAISGVDKMDSTSANVDVPDFLSSLTGDIKGLKIAVPKEYLGEGVGKEARESVLAALKVLEGLGATWEEVSLPHSKYALATYYLLSSSEASANLARFDGIRYGYRTDNADNLIDLYKQTRAEGFGNEVKRRIMLGTFALSSGYYDAYYKKAQKVRTLIKKDFEDVFEKYDVIVGPTTPTPAFKIGENTKDPLTMYANDILTIPVNLAAYRESGAMRLADGLPLGLQIIGKHFDEALYTALLMHLNKQETIIKQNLNCKG

SEQ ID NO: 5 - DNA, 1431 bp, Bacillus subtilis
  CDS (1)..(1428): subunit B, corresponds to nucleotides 1876 - 3306 of SEQ ID NO 1
ttgaactttgaaacggtaatcggacttgaagtccacgttgagttaaaaacaaaatcaaaaattttctcaagctctccaacgccattcggcgcggaggcgaatacgcagacaagcgttattgacctcggatatccgggcgtcctgcctgttctgaacaaagaagccgttgaattcgcaatgaaagccgctatggcgctcaactgtgagatcgcaacggatacgaagtttgaccgcaaaaactatttctatcctgacaacccgaaagcgtatcagatttctcaatttgataagccaatcggcgaaaacggctggatcgaaattgaagtcggcggcaaaacaaaacgcatcggcatcacgcgccttcatcttgaagaggatgccggaaaactgacgcatacgggcgacggctattctcttgttgacttcaaccgtcaaggaacgccgcttgttgagtncgtatcagagccggacatccgcacgccggaagaancgtacgcatatcttgaaaagctgaaatccatcatccaatatacaggcgtttctgactgtaaaatggaagaaggctcacttcgctgtgacgccaatatctctcttcgtccgatcggccaagaggaattcggcacaaaaacagaattgaaaaacttgaactcctttgcgtttgttcaaaaaggccttgagcatgaagaaaaacgccaggagcaggttcttctttccggcttcttcatccagcaagaaactcgccgttatgatgaagcaacgaagaaaaccattcttatgcgtgtcaaagagggatctgacgactaccgttactttccagagccagatctagtcgagctctacattgatgatgaatggaaggaacgcgtaaaagcaagcattcctgagcttccggatgagcgccgcaagcgttatatcgaagagcttggcttcgctgcatatgacgcaatggttctgacgctgacaaaagaaatggctgatttcttcgaagaaaccgttcaaaaaggcgctgaagctaaacaagcgtctaactggctgatgggtgaagtgtcagcttacctaaacgcagaacaaaaagagcttgccgatgttgccctgacacctgaaggccttgccggcatgatcaaattgattgaaaaaggaaccatttcttctaagatcgcgaagaaagtgtttaaagaattgattgaaaaaggcggcgacgctgagaagattgtgaaagagaaaggccttgttcagatttctgacgaaggcgtgcttctgaagcttgtcactgaggcgcttgacaacaatcctcaatcaatcgaagactttaaaaacggaaaagaccgcgcgatcggcttcctagtcggacagattatgaaagcgtccaaaggacaagccaacccgccgatggtcaacaaaattctgcttgaagaaattaaaaaacgctaa

SEQ ID NO: 6 - protein, 476 aa, Bacillus subtilis
  MISC_FEATURE (152)..(152): Xaa is Phe, Ser, Tyr, or Cys
LNFETVIGLEVHVELKTKSKIFSSSPTPFGAEANTQTSVIDLGYPGVLPVLNKEAVEFAMKAAMALNCEIATDTKFDRKNYFYPDNPKAYQISQFDKPIGENGWIEIEVGGKTKRIGITRLHLEEDAGKLTHTGDGYSLVDFNRQGTPLVEXVSEPDIRTPEEXYAYLEKLKSIIQYTGVSDCKMEEGSLRCDANISLRPIGQEEFGTKTELKNLNSFAFVQKGLEHEEKRQEQVLLSGFFIQQETRRYDEATKKTILMRVKEGSDDYRYFPEPDLVELYIDDEWKERVKASIPELPDERRKRYIEELGFAAYDAMVLTLTKEMADFFEETVQKGAEAKQASNWLMGEVSAYLNAEQKELADVALTPEGLAGMIKLIEKGTISSKIAKKVFKELIEKGGDAEKIVKEKGLVQISDEGVLLKLVTEALDNNPQSIEDFKNGKDRAIGFLVGQIMKASKGQANPPMVNKILLEEIKKR

SEQ ID NO: 7 - DNA, 291 bp, Bacillus subtilis
  CDS (1)..(288): Subunit C, corresponds to nucleotides 103 - 393 of SEQ ID NO 1
atgtcacgaatttcaatagaagaagtaaagcacgttgcgcaccttgcaagacttgcgattactgaagaagaagcaaaaatgttcactgaacagctcgacagtatcatttcatttgccgaggagcttaatgaggttaacacagacaatgtggagcctacaactcacgtgctgaaaatgaaaaatgtcatgagagaagatgaagcgggtaaaggtcttccggttgaggatgtcatgaaaaatgcgcctgaccataaagacggctatattcgtgtgccatcaattctggactaa

SEQ ID NO: 8 - protein, 96 aa, Bacillus subtilis
MSRISIEEVKHVAHLARLAITEEEAKMFTEQLDSIISFAEELNEVNTDNVEPTTHVLKMKNVMREDEAGKGLPVEDVMKNAPDHKDGYIRVPSILD